(19) United States
(12) Patent Application Publication
(10) Pub. No.: US 2024/0049995 A1
(43) Pub. Date: Feb. 15, 2024

(54) Methods and Devices for Inducement of Sweat for Medical Diagnostics

(71) Applicants: Georgia Tech Research Corporation, Atlanta, GA (US); Emory University, Atlanta, GA (US); Children's Healthcare of Atlanta, Inc., Atlanta, GA (US)

(72) Inventors: Song Li, Atlanta, GA (US); Lokesh Guglani, Alpharetta, GA (US); Mark R. Prausnitz, Atlanta, GA (US)

(21) Appl. No.: 18/269,815

(22) PCT Filed: Dec. 30, 2021
(86) PCT No.: PCT/US2021/065760

§ 371 Date: Jun. 27, 2023

Related U.S. Application Data
(60) Provisional application No. 63/132,086, filed on Dec. 30, 2020.

(51) Int. Cl. A61B 5/145; A61B 5/00; A61B 10/00; A61K 31/4178; A61K 9/00; A61K 47/34; A61K 47/26

(52) U.S. Cl. CPC A61B 5/14521; A61B 5/685; A61B 10/0064; A61K 31/4178; A61K 9/0021; A61K 47/34; A61K 47/26

(57) ABSTRACT

Methods and devices are provided for sweat inducement, which is useful in diagnostics, such as diagnosis of cystic fibrosis in a patient. The method includes applying a microneedle patch, which comprises microneedles which comprise pilocarpine or another sweat-inducing agent, to the skin of the patient effective to cause the microneedles to penetrate across the epidermis and into the dermis releasing the pilocarpine or another sweat-inducing agent into the skin in an amount effective to induce secretion of sweat from the skin. The secreted sweat can be collected and analyzed, for example by measuring chloride concentration in the sweat, which may be indicative of cystic fibrosis.

### CROSS REFERENCE TO RELATED APPLICATIONS

This application claims benefit of U.S. Provisional Application No. 63/132,086, filed Dec. 30, 2020, which is incorporated herein by reference.

### BACKGROUND

This invention is generally in the field of physiological metrics measurements, including but not limited to medical diagnostics, and more particularly to methods for inducing sweat for diagnostic testing, for example, for cystic fibrosis.

Conventional testing for cystic fibrosis (CF) in patients involves the use of iontophoresis to deliver pilocarpine into the skin to induce sweating, followed by collecting and testing of the sweat. This has been the standard clinical technique since the 1960s. Since the 1980s, the technique has included application of an agar disk containing pilocarpine onto a patient's arm and using an iontophoresis device to drive the pilocarpine from the disk into the skin over the course of about 5 minutes, and then a sweat collector is applied to the patient's arm to collect sweat over about 30 minutes.

All newborn infants in the United States are routinely screened for CF, since early detection and treatment of CF is beneficial to long-term outcomes of those affected. For infants with a positive newborn screening test for CF, the sweat test is the next step to confirm the diagnosis, as the measurement of sweat chloride concentration in sweat remains the gold standard for the diagnosis of CF. However, in many instances, inadequate volumes of sweat are collected, necessitating repeat testing. The failure of adequate sweat collection is especially common when the test is performed on infants less than 3 months of age. These delays cause significant anxiety for parents of the newborn who are waiting to learn whether their child has CF. The delay in diagnosis also undesirably delays initiation of treatment for CF for those persons who are determined to have CF.

Accordingly, there is an urgent need to develop more accessible and simple-to-administer alternatives for inducing and collecting sweat. Such methodology will facilitate expedient and accurate diagnosis of CF in infants. There also remains a need for improved methods and devices for inducing sweating for medical and non-medical applications, including but not limited to screening and diagnoses of diseases, disorders, and conditions that may be detectable from a person's sweat.

### BRIEF SUMMARY

In one aspect, a method for inducing sweat secretion from a patient's skin is provided. The method includes applying a microneedle patch, which comprises microneedles which comprise a sweat-inducing agent, to the skin of the patient effective to cause the microneedles to penetrate across the epidermis and into the dermis; and releasing the sweat-inducing agent into the skin in an amount effective to induce secretion of sweat from the skin.

In another aspect, a diagnostic method is provided that includes inducing secretion of sweat from a patient's skin using a microneedle patch; and then analyzing the sweat for the presence, absence, or concentration of one or more analytes.

In still another aspect, a microneedle patch is provided. The patch includes a support layer; and an array of microneedles extending from the support layer, wherein the microneedle patch is configured for application to a patient's skin and the microneedles comprise a sweat-inducing agent, such as a cholinergic agonist, such as pilocarpine.

In yet another aspect, a method of diagnosis of cystic fibrosis in a patient is provided. The method includes applying a microneedle patch, which comprises microneedles which comprise pilocarpine, or another sweat-inducing agent, to the skin of the patient effective to cause the microneedles to penetrate across the epidermis and into the dermis; releasing the pilocarpine, or other sweat-inducing agent, into the skin in an amount effective to induce secretion of sweat from the skin; collecting a volume of the sweat secreted from the skin; and analyzing the collected sweat for an analyte indicative of cystic fibrosis.

### BRIEF DESCRIPTION OF THE DRAWINGS

FIG. 1 is a perspective view of a microneedle patch according to one embodiment of the present disclosure.

FIGS. 2A-2B are microphotographs of a single microneedle. The microneedle is shown before (FIG. 2A) and after (FIG. 2B) it is applied to skin. Scale bar 0.5 mm.

FIG. 3 depicts an array of microneedle patch-generated micropores created in skin after the application of a microneedle patch. Scale bar 5 mm.

FIG. 4 is a graph showing data from one example, comparing total volume of sweat collected after inducement by pilocarpine delivery by microneedle patches as described herein or by conventional iontophoresis.

FIG. 5 is a graph showing data from one example, comparing sweat volume collected per unit of pilocarpine dose after inducement by pilocarpine delivery by microneedle patches as described herein or by conventional iontophoresis.

FIG. 6 is a graph showing data from one example, comparing sweat volume collected per unit of skin area after inducement by pilocarpine delivery by microneedle patches as described herein or by conventional iontophoresis.

FIG. 7 is a graph showing data from one example, comparing chloride content of collected sweat after inducement by pilocarpine delivery by microneedle patches as described herein or by iontophoresis.

### DETAILED DESCRIPTION

New and improved methods and devices have been developed for inducing sweat secretion from skin for medical diagnostics purposes. In particular embodiments, the method includes (i) applying a microneedle patch, which comprises microneedles which comprise a sweat-inducing agent, such as a cholinergic agonist, to the skin of the patient effective to cause the microneedles to penetrate across the epidermis and into the dermis; and (ii) releasing the sweat-inducing agent into the skin in an amount effective to induce secretion of sweat from the skin. In a preferred embodiment, the cholinergic agonist comprises pilocarpine.

The microneedle patch enables sweat secretion inducement in a minimally invasive, painless, and convenient manner. Thus, the devices and methods herein can make sweat testing simpler and more widely available than current iontophoresis-based methods.

In fact, it is a particular advantage of the present methods that iontophoresis is not required. Accordingly, no electrical current is applied to the skin, which eliminates the risk of skin burns associated with the conventional iontophoresis-driven administration of the sweat-inducing agent into the patient's skin.

Furthermore, in at least some embodiments, the present methods may enable higher sweat output per unit area of skin, as compared to methods utilizing conventional administration of pilocarpine from agar disks using iontophoresis. In fact, as detailed in the examples, the amount of pilocarpine delivered per unit area of skin may be up to approximately twice as large after microneedle patch administration compared to administration by iontophoresis.

The term “patient” refers to any person (human) to whom the sweat inducement methods are applied. The term “patient” includes but is not limited to a person in need of medical care or a person in need of other physiological assessments. The patient may be an infant, child, or adult.

New and improved diagnostic methods are also provided that include (i) inducing secretion of sweat from a patient's skin as described herein; and (ii) analyzing the sweat for the presence, absence, or content of one or more analytes. That is, the induced sweat, or the collected sweat, may be analyzed for various analytes in the sweat, e.g., by detecting, measuring, and/or determining the presence and/or amounts of an analyte of interest, for example, for determining or monitoring of one or more physiological or pathological conditions or attributes in the patient.

The Methods

In some embodiments, the methods include applying a microneedle patch, which comprises microneedles which comprise pilocarpine (or another sweat-inducing agent) to the skin of the patient effective to cause the microneedles to penetrate across the epidermis and into the dermis; releasing the pilocarpine (or other sweat-inducing agent) into the skin in an amount effective to induce secretion of sweat from the skin; and then analyzing the sweat for a specific analyte. The method may include collecting a volume of the sweat secreted from the skin and the analyzing is carried out on the collected sweat. In a particular embodiment, the analyte is one indicative of a disease. In a particular example, the analyte is chloride concentration, which is indicative of cystic fibrosis.

In some embodiments, the step of applying a microneedle patch comprises manually pressing the microneedle patch against the patient's skin. For example, the microneedle patch may be applied to an area of the patient's arm (e.g., forearm) or leg. The application site preferably is sanitized prior to application of the microneedle patch, for example using a conventional alcohol wipe. If needed, the application site may be allowed to dry before application of the microneedle patch. The patch then is applied to the patient's skin using a sufficient pressure to have the microneedles penetrate across the epidermis and into the dermis.

In some embodiments, the methods further include removing the microneedle patch from the skin after a period of time effective to release the pilocarpine (or other sweat-inducing agent) from the microneedle patch into the patient's skin. In some embodiments, the methods include removing the microneedle patch from the skin in a manner effective to separate the microneedles from a support layer of the microneedle patch, wherein the separated microneedles remain in the patient's skin and dissolve to release the pilocarpine (or other sweat-inducing agent). For example, the microneedles may break off the patch backing immediately upon application to the skin, so that the patch backing may promptly thereafter be removed from the skin. Accordingly, in various embodiments of these methods, the period of time may be between 1 second and 15 minutes. The period may be, for example, between 1 second and 10 minutes, between 1 second and 1 minute, between 10 seconds and 10 minutes, between 10 seconds and 1 minute, between 1 minute and 15 minutes, between 1 minute and 10 minutes, or about 5 minutes.

In some embodiments, the skin-embedded microneedles, whether still connected to the backing or separated from it, release the pilocarpine (or other sweat-inducing agent) by dissolution of the microneedles in the aqueous fluid of the skin tissues. Accordingly, in some preferred embodiments of the methods, the microneedles are dissolvable microneedles as described below in the Microneedle Patch section.

In some other embodiments, the pilocarpine (or other sweat-inducing agent) is associated with, and released from, the microneedles by different mechanisms than foregoing dissolvable microneedles. In one such example, the pilocarpine (or other sweat-inducing agent) is coated onto microneedles made of essentially any suitable material, including non-water soluble materials. In another example, the microneedles are hydrogels that swell in the skin and release the pilocarpine (or other sweat-inducing agent) from within the hydrogel. In still another example, the microneedles are not hydrogels or water-soluble and include hollow or porous structural portions, and the pilocarpine (or other sweat-inducing agent) is loaded over the cavities or pores of those hollow or porous structural portions and released therefrom following insertion into the skin.

In some embodiments, the method is effective to deliver from 250 μg to 1500 μg of pilocarpine (or other sweat-inducing agent) per cm² of skin. In some embodiments, the method is effective to deliver from 500 μg to 1000 μg of pilocarpine (or other sweat-inducing agent) per cm² of skin. In some embodiments, the method is effective to deliver at least 250 jig, at least 300 μg, at least 400 μg, at least 500 μg, at least 600 μg, at least 700 μg, or at least 800 μg of pilocarpine (or other sweat-inducing agent) per cm² of skin.

In some embodiments, a total of more than 1.38 mg pilocarpine is administered into the skin. For example, the microneedle patch may deliver 1.4 or 1.5 mg or more of the pilocarpine to the skin of the patient. In one non-limiting example, the microneedle patch delivers from 1.50 mg to 2.50 mg of pilocarpine.

In some embodiments, the collecting of the sweat includes applying an absorbent material to the skin or positioning a collection tube at the skin surface to permit sweat to be drawn into a bore in the tube, for example, by capillary action. The absorbent material may be a woven or non-woven fibrous material, such as a cotton swab or gauze, or porous structure, such as a sponge. Capillary collection tubes are known in the art. For example, the collection tube may be part of a Macroduct™ Sweat Collector. In some embodiments, the sweat may be collected in the microneedle patch itself.

The amount of sweat collected generally should be any amount of sweat that is suitable for the analytical method to be used. In some embodiments, the volume of sweat collected is from 5 μl to 150 μl. For example, the collected volume may be from 10 μl to 100 μl. In some embodiments, the volume of sweat collected may be from 15 μl to 30 μl. In one embodiment, a total of at least 17 μl of sweat may be induced by the microneedle patch and collected.

In some embodiments, the volume of sweat collected is between the minimum volume that is effective for chloride concentration measurements by a current or future technique of chloride measurement and a maximum that collectable from the skin over a 30-minute collection period.

In some embodiments, the sweat collected per area of skin into which the pilocarpine (or other sweat-inducing agent) is released is from 2 μl per cm² to 50 μl per cm². In some embodiments, the sweat collected per area of skin into which the pilocarpine is released is at least 2.6 μl per cm². In some other embodiments, the sweat collected per area may be from 10 μl per cm² to 40 μl per cm². In some embodiments, the sweat collected per area is at least 15 μl per cm², or at least 20 μl per cm².

The collected sweat can be analysed by any suitable method for any analytes. For example, it may undergo chloride analysis with a chloridometer or total electrolyte analysis for example, using a Sweat-Chek Analyzer™. Other analyses also are envisioned, such as skin-interfacing microfluidic devices known in the art. See, e.g., Ray, et al., Science Translational Medicine, 31 Mar. 2021, Vol 13, Issue 587.

The presently disclosed microneedle patch configured to induce sweating can be used in clinical settings, in personal health monitoring, or in other applications, such as non-medical context, e.g., athletic performance assessment, military readiness assessment, etc. The microneedle patch advantageously may replace conventional sweat-inducing techniques that involve hypodermic injections and/or iontophoresis, because the microneedle patch is much easier to use. Because of the relative simplicity of its use, the microneedle patch can also be used by any person after brief training for personal health monitoring, e.g., at home.

Cystic Fibrosis Testing

The methods described herein are particularly useful to produce and collect a sweat sample that can be used in a better tool in diagnosing cystic fibrosis. In a preferred embodiment, the chloride concentration in the collected sweat is quantified for the diagnosis of cystic fibrosis using a chloridometer or other conventional instruments. As known in the art, elevated chloride levels in sweat are indicative of cystic fibrosis.

The presently disclosed pilocarpine-containing microneedle patches offer a simple and more accessible alternative for sweat induction to support efficient and minimally invasive cystic fibrosis diagnosis in infants and children. In one particular embodiment, the microneedle patch is applied to the skin of an infant, for example on the arm, after the infant has a positive CF screening. Pilocarpine then is released from microneedles of the patch into the infant's skin effective to induce secretion of sweat, and then a volume of the sweat secreted is collected from the skin using conventional means, such as the Macroduct™ Sweat Collector. The collected sweat is then analyzed by measuring the chloride concentration in the collected volume of sweat using a chloridometer as known in the art.

The larger pilocarpine dose per unit area enabled by the present microneedle patch delivery methods compared to conventional iontophoresis methods may facilitate more consistently generated amount of sweat required to perform a chloride measurement, thus potentially making the sweat test more reliable and avoiding the need for repeated measurement attempts experienced with conventional methods.

The Microneedle Patch

In embodiments, the microneedle patch useful in the present methods includes a support layer, and an array of microneedles extending from the support layer, wherein the microneedle patch is configured for application to a patient's skin and the microneedles include a sweat-inducing agent. The sweat-inducing agent may be cholinergic agonist, such as pilocarpine.

As used herein, the term “pilocarpine” refers to (3S,4R)-3-ethyl-4-((1-methyl-1H-imidazol-5-yl)methyl)dihydrofuran-2(3H)-one, and pharmaceutically acceptable salts, and/or solvates, thereof. In the case of sweat collection for measurements of chloride content, the HCl or other chloride-containing salt form of pilocarpine would not be used because chloride from the pilocarpine salt could affect chloride concentrations measured in the collected sweat. In some preferred embodiments, the pilocarpine is pilocarpine nitrate.

In some other embodiments, the sweat-inducing agent may be selected from suitable drugs known in the art to cause excess perspiration or sweating as a side effect. See, e.g., https://www.sweathelp.org/pdf/drugs_2009.pdf. In one embodiment, the sweat-inducing agent is carbachol.

The sweat-inducing agent is part of the microneedle structure. For example, the sweat-inducing agent may be dispersed in a matrix material forming at least part of the microneedle structure, part of a coating material on the microneedle, or a combination thereof.

In a preferred embodiment, the microneedles are dissolvable. As used herein, the term “dissolvable” means that the microneedles include water-soluble materials which dissolve in water in the skin, following insertion of the microneedles. The dissolution should be at rate useful to release the sweat-inducing agent into tissues of the skin at a practical, or clinically useful, rate. In a preferred embodiment, the microneedles are formed of the sweat-inducing agent dispersed in one or more water-soluble matrix materials.

In some other embodiments, it may be desirable to induce continuous sweating over an extended period of time, for sweat collection and analyte measurement over an extended period. In such cases, the microneedles may be configured to slowly release the sweat-inducing agent into the skin, for example, by using any of the mechanisms known in the art for controlled, sustained drug delivery from microneedles. In some embodiments, this is accomplished by making the microneedles of a composition that includes the sweat-inducing agent (e.g., pilocarpine) and one or more biomaterials selected from hydrogels, biodegradable polymers (e.g., PLGA), non-degradable polymers, and the like.

The microneedles may include a variety of suitable biocompatible, water-soluble matrix materials. The matrix materials, in combination with the sweat-inducing agent, should impart the necessary mechanical strength for reliable insertion of the microneedles into the skin. Generally, the sweat-inducing agent is included in a stable composition (forming the microneedles) in which the sweat-inducing agent therein essentially retains its physical stability and/or chemical stability and/or biological activity upon storage. The matrix materials may be selected from pharmaceutically acceptable excipients known in the art.

In some preferred embodiments, the matrix material of the microneedles comprise two or more matrix materials. In some embodiments, the matrix material may include or consist of a combination of a poly(vinyl alcohol) (PVA) and a disaccharide. Examples of disaccharide include sucrose, lactose, and maltose. For example, the matrix material may include PVA and sucrose. In some other embodiments, other water soluble polymers are used in place of or in combination with PVA.

In some embodiments, the fraction of the sweat-inducing agent in the microneedles ranges from 20% to 60% by weight. In some embodiments, the microneedles comprise from 30% to 50% by weight pilocarpine. In some sub-embodiments, these microneedles comprise from 70% to 50% by weight a mixture of a PVA and a disaccharide, such as sucrose. In some other embodiments, the microneedles are 20-60% by weight pilocarpine, and the other materials are non-water soluble materials that are formed in a porous or hollow structure, where the pores or hollow portion(s) of the microneedle contain the pilocarpine.

In one embodiment, the microneedles comprise about 40% by weight pilocarpine nitrate. In some sub-embodiments, the microneedles comprise about 60% by weight a mixture of a PVA and a disaccharide, such as sucrose.

The microneedles may have any suitable shape. In some embodiments, the microneedles are conical. In some other embodiments, the microneedles may be blade-like, or pyramidal. In some embodiments, the microneedles have a straight proximal portion and a tapered distal portion. The shaft of the microneedle may have a circular, oval, or polygonal cross-sectional shape.

The microneedle patch is constructed to administer to the skin an amount of the sweat-inducing agent across an area of skin effective to induce secretion of sweat in a total volume that is required for a particular analysis. This may be controlled for example by selecting/adjusting the amount of the amount of the sweat-inducing agent releasable from each microneedle, the total number of microneedles in the patch array, and/or the spacing of the microneedles/size of the patch. In some embodiments, the microneedle patch is configured to deliver at least 240 μg of pilocarpine per cm² of patient's skin. In some embodiments, the microneedle patch is configured to deliver at least 250 μg of pilocarpine per cm² of patient's skin.

The microneedles may have a length between 200 μm and 2,000 μm. In some embodiments, the microneedles have a length between 500 μm and 1,000 μm. For example, the microneedles may have a length of about 600 μm, about 700 μm, about 800 μm, or about 900 μm.

The area of the microneedle patch may be any suitable dimensions. In some embodiments, the area is between 0.5 cm² and 10 cm². In some embodiments, the area is from 2 cm² to 8 cm². In some embodiments, the area is from 5 cm² to 6 cm². In one example, the area is 5.8 cm². Other dimensions are envisioned.

The microneedles have a base (or proximal) end and an opposing (distal) tip end. The base end of each microneedle is attached, directly or indirectly, to the support layer (or base substrate) of the microneedle patch. In some preferred embodiments, the microneedle patch further includes base pedestals between and connecting the support layer and each of the microneedles. The base pedestals may be made of a polymeric material, such as PVA. In some embodiments, the base pedestals have a height between 200 μm and 800 μm. In some embodiments, these microneedles are coated with a formulation containing pilocarpine.

In some embodiments, the sweat-inducing agent is located only in the microneedles, e.g., predominately at the tip end portion of the microneedle, and not in the support layer. In some other embodiments, the sweat-inducing agent may be dispersed in the support layer too, for example, wherein the support layer and microneedles are fabricated of the same materials. In some embodiments, the pilocarpine is located predominantly or exclusively in a coating on the microneedles.

The microneedle array may have a variety of shapes, including circular or square. In some embodiments, the size of the microneedle patch is between 1 cm and 10 cm in its longest dimension. In some embodiments, the microneedle patch includes from 100 to 1000 microneedles.

In some embodiments, the microneedle patch include a handle, or tab, for manipulating the patch.

In some embodiments, the microneedle patch comprises a pressure-sensitive adhesive suitable for temporarily securing the patch to the skin.

In some embodiments, the microneedle patch includes a feedback indicator configured to inform the user that the microneedles have penetrated the skin and/or that the sweat-inducing agent has been released into the skin.

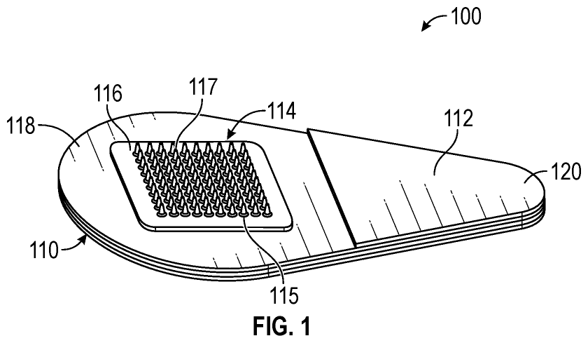

One embodiment of a microneedle patch 100 is shown in FIG. 1. The microneedle patch 100 includes a microneedle array 114 extending from a support layer 116. The microneedles 117 extend from a base pedestal 115. The support layer 116 is affixed to adhesive layer 118 of a handling structure 110 that includes a tab portion 112 and an adhesive cover 120. Other configurations of handling structures are envisioned, some of which are described in U.S. Pat. No. 10,265,511, which is incorporated herein by reference.

The microneedle patches may be made a process that include molding microneedles as described in U.S. Pat. No. 10,828,478, which is incorporated herein by reference.

The present invention may be further understood with reference to the following non-limiting examples.

### EXAMPLES

Experiments were conducted to evaluate whether microneedle patches could be used to perform sweat tests and to evaluate whether microneedle patches can be used as an alternative to iontophoresis to administer pilocarpine to induce sweating, including for use in the diagnosis of cystic fibrosis.

All data are presented as mean±standard deviation. Total sweat volume, sweat volume/drug dose, sweat volume/skin area, and sweat chloride concentration were compared between microneedle and iontophoresis sites with two-tailed unpaired Student's t-tests. Statistical significance was set at p≤0.05 for all comparisons.

### Example 1: Microneedle Patch with Microneedles Comprising Pilocarpine

Pilocarpine-loaded microneedle patches were fabricated by a two-step molding process using polydimethylsiloxane (PDMS) molds based on an established method. The first casting solution was a mixture of 10% (w/v) pilocarpine nitrate, 10% (w/v) poly(vinyl alcohol) (PVA) and 5% (w/v) sucrose, which was prepared in deionized water. This solution was cast on PDMS molds under vacuum to facilitate filling the solution into the mold cavities to form the microneedles. After 20 min, excess solution was removed, and the filled molds were centrifuged at 5000 g for 20 min to dry the drug-loaded microneedles. The second casting solution containing 20% (w/w) polystyrene in 1,4-dioxane was then cast on the filled PDMS molds under vacuum to form the patch backing. The molds were kept under vacuum for another 3 h to dry the solution at room temperature, and then further dried at 40° C. overnight before demolding the microneedle patches using adhesive tapes.

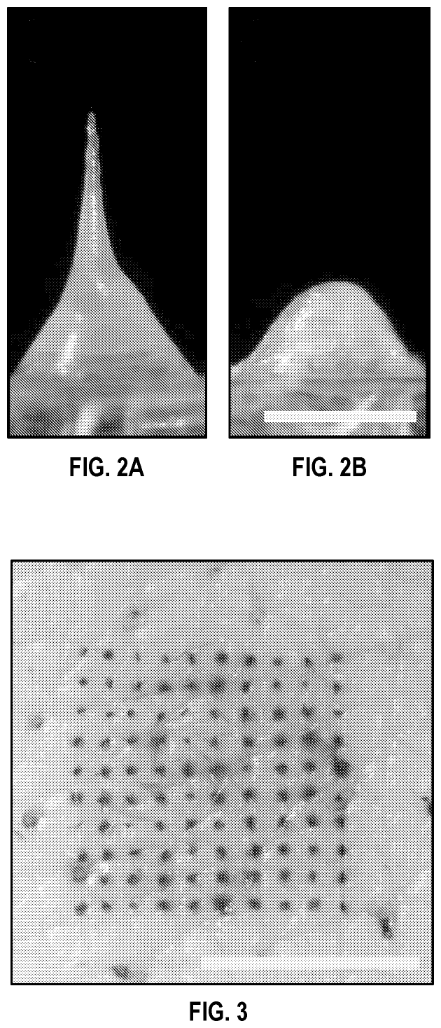

Each microneedle patch consisted of a 10×10 array of the microneedles arranged within a square with approximately 7 mm sides (i.e., ˜0.5 cm²). As shown by microscopic examination (see FIG. 2A), each conical microneedle (base diameter 200 μm, height 600 μm) was mounted atop an wider pedestal (base diameter 600 μm, height 400 μm).

The solid microneedles were composed of 40% by weight pilocarpine, 40% by weight PVA, and 20% by weight sucrose. The total amount of pilocarpine loaded per microneedle patch was measured as 500±48 μg (n=4). The PVA provided the mechanical strength the microneedle needs to penetrate the skin, and the sucrose facilitated microneedle dissolution in the skin following the insertion into the skin.

### Example 2: Ex Vivo Application of Pilocarpine Microneedles to Porcine Skin

Microneedle patches from Example 1 were applied to shaved porcine skin ex vivo to study their skin insertion properties before application on horses in vivo. A microneedle patch was manually pressed against the porcine skin by thumb for ˜10 s, and then left in place for 20 min to allow microneedle dissolution and release of drug in the skin. After being removed from the skin, the patches were saved for further examination.

After application to porcine skin ex vivo, the microneedles dissolved in the skin, leaving only the base pedestals (FIG. 2B), indicating that the pilocarpine loaded in the microneedles was successfully delivered into the skin during microneedle patch application. Treating the skin with a dye that selectively stains sites of skin puncture revealed an array of microneedle-generated micropores with the same 10×10 array geometry as the microneedle patch (FIG. 3), further indicating the ability of microneedles to penetrate the skin.

After application to porcine skin ex vivo, the residual pilocarpine content per microneedle patch was 237±73 μg (n=6), indicating that the delivered dose was ˜263 μg (i.e., ˜526 μg/cm²) and the delivery efficiency was ˜53%.

The pilocarpine dose delivered by microneedle patches was calculated as the difference between the pilocarpine contents in patches before and after application to skin. The pilocarpine content in microneedle patches was measured by HPLC after dissolving the patch in a known volume of deionized water.

### Example 3: Ex Vivo Application of Iontophoretic Pilogel Discs to Porcine Skin

The commercially available iontophoretic Pilogel discs were circular with a diameter of 2.72 cm (i.e., ˜5.8 cm²), thereby contacting an area of skin more than 10 times larger than the microneedle patch. The iontophoretic Pilogel discs therefore had much higher pilocarpine loading amount, measured as 15.94±0.27 mg (n=3). After iontophoresis on porcine skin for 10 min, the used discs contained 14.56±0.15 mg (n=3) residual pilocarpine, which indicates the delivered dose by iontophoresis was ˜1.38 mg (i.e., ˜238 μg/cm²) and the delivery efficiency was 8.7%. The delivery efficiency of iontophoresis was significantly lower than that of the microneedle patch.

### Example 4: In Vivo Application of Microneedle Patches and Iontophoresis to Induce Sweating in Horse Model

Eight healthy outbred adult horses were used as the animal model. Prior to all testing, the cervical region of the skin of the horses was shaved to permit good contact of the microneedle patches, iontophoretic pilocarpine discs and sweat collection pads to the skin.

Procedure

Pilocarpine-induced sweat production via microneedle patches and iontophoresis was then compared in 4 horses after acclimatization. In each animal, three microneedle patches from Example 1 above were applied manually by thumb pressure to the right side of the neck, approximately 5 cm apart, and left in place for 20 min. Concurrently, on the left side of the neck, Pilogel Iontophoretic Discs were mounted onto electrodes, and pilocarpine was delivered via iontophoresis for 10 min (2 machine cycles) in two separate locations sequentially.

After completion of iontophoresis and removal of the microneedle patches, sweat was collected from 25 sites on the neck of 4 horses to quantify volume and chloride concentration. In pilot studies, the conventional Macroduct® Sweat Collector used for sweat collection in humans could not be consistently adhered to the convex cervical region on the horse, leading to unreliable and inconsistent sweat collection. Thus, a modified sweat collection protocol was developed using single layer cotton gauze pads covered by a similarly-sized piece of 150 μm-thick polypropylene plastic sheeting and secured under a piece of heavy-duty adhesive tape (approximately 5×15 cm). Gauze pads to collect microneedle-induced and iontophoresis-induced sweat were 1 cm² and 2 cm², respectively. Plastic sheeting approximately 1 mm larger in length and width was applied over the gauze pads. After 30 min, the gauze pads were collected and immediately weighed on a microbalance to calculate sweat volume by subtracting the dry weight. Gauze pads were then immediately placed inside a 3 ml polypropylene syringe barrel inserted in a 15 ml conical polypropylene tube and sealed prior to centrifuging at 1100×g for 10 min. Sweat recovered after centrifugation was collected into a polypropylene microcentrifuge tube and frozen at −80° C. until analysis for chloride concentration.

Results

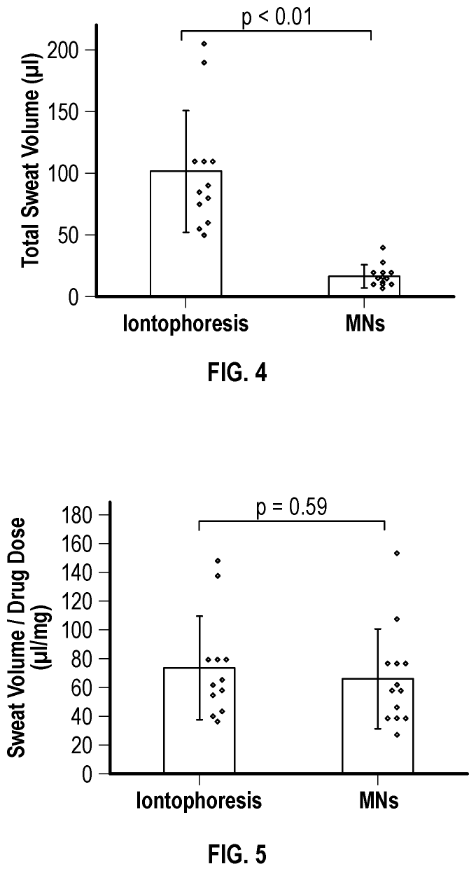

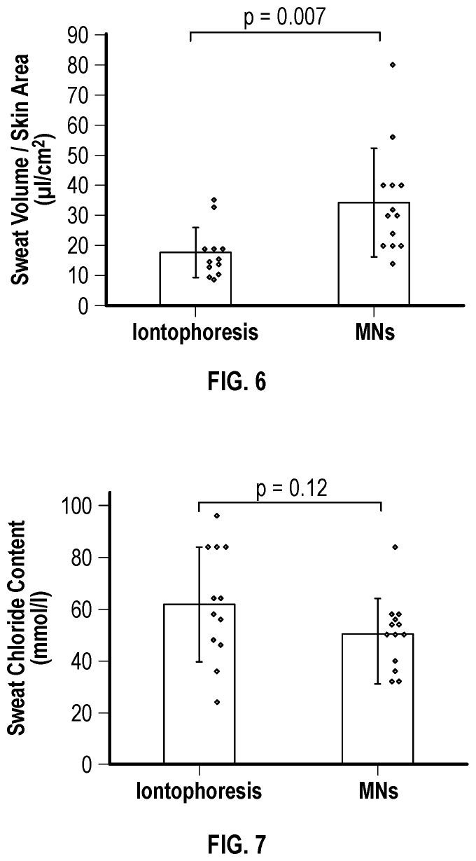

From all application sites, at least 10 ul of sweat was collected. The average total sweat volume from an iontophoresis site was 101±49 ul over a pilocarpine application area of 5.8 cm², corresponding to a sweat collection density of 17±8 μl/cm². The average total sweat collected from a microneedle patch site was 17±8 ul over a pilocarpine application area of 0.5 cm², corresponding to a sweat collection density of 34±16 μl/cm² (FIG. 4 and FIG. 6). Sweat density is an appropriate basis for comparison between the two techniques because sweat production is expected to scale directly with area and because sweat collection is usually done over a standard area of skin using a sweat collection device.

While the total amount of sweat collected from the iontophoresis sites was greater than that collected from the microneedle patch sites (FIG. 4), when accounting for the different pilocarpine application areas, the sweat collection density from the microneedle patch sites was 2.0-fold greater than that collected from the iontophoresis sites (FIG. 6). This ratio was relatively consistent on each of four horses (2.3, 1.6, 2.1 and 1.6-fold greater). This suggests that the difference between microneedle patches and iontophoresis on sweat induction was not determined by the individual differences between horses. Instead, the difference in sweat collection density appears to mainly reflect the different sweat-inducing abilities of the two pilocarpine delivery procedures.

The sweat volume per unit of pilocarpine dose delivered to the skin was calculated. This analysis revealed no significant difference between iontophoresis (73±36 μl/mg) and microneedle patches (66±34 μl/mg) (FIG. 5). However, because the amount of pilocarpine delivered per unit area of skin was 2.2-fold greater when administered by microneedle patches (˜526 μg/cm²) compared to iontophoresis (˜238 μg/cm²), this likely accounts for the greater sweat collection density seen after pilocarpine delivery by microneedle patch. Thus, using microneedle patches to deliver pilocarpine has a comparable or better sweat-inducing capability as the traditional iontophoresis.

It should be noted that although sweat collection density was greater using a microneedle patch, the microneedle patch induced less total sweat volume than iontophoresis. Because the microneedle patch delivered twice as much pilocarpine per unit area, a larger microneedle patch with the same area as the pilocarpine disc used for iontophoresis (i.e., 5.8 cm²) should correspondingly deliver twice as much pilocarpine and thereby induce more total sweat volume compared to iontophoresis, because sweat production is known to scale with pilocarpine dose delivered.

Chloride contents in the iontophoresis-induced sweat (60.0±21.8 mmol/L) and microneedle patch-induced sweat (50.3±13.8 mmol/L) were not significantly different (FIG. 7), indicating that the method of pilocarpine administration (iontophoresis vs. microneedle patch) did not significantly affect the chloride content in the collected sweat.

### Example 5: Further Comparisons of Microneedle Patches and Iontophoresis

When the microneedle patches were applied to the skin on horses in vivo, more pilocarpine, 407±46 μg (n=13), was dissolved from the microneedle patches compared with the ex vivo measurements in porcine skin. An explanation for this difference may be that the sweat induced by the microneedle patch in the horse (but not in the porcine skin ex vivo) might have further dissolved the microneedles at the skin surface, giving the appearance of greater pilocarpine delivery efficiency. The same effect might have also occurred at the iontophoresis sites. The analysis of the results was based on the ex vivo data on the delivered pilocarpine dose from both microneedle patches (Example 1, square with side length of ˜0.7 cm) and iontophoresis (Pilocarpine Iontophoresis Disc, round with a diameter of ˜2.72 cm) as 0.26 mg and 1.38 mg, respectively as shown in Table 1.

TABLE 1
Comparison of parameters between microneedle patches and iontophoresis

| | | Pilocarpine |
|---|---|---|
| | Microneedle patches | Iontophoresis Discs |
| Application area (cm²) | 0.5 | 5.8 |
| Pilocarpine dose (mg)^{c} | 0.26 ± 0.07 | 1.38 ± 0.15 |
| Pilocarpine/application area | 0.52 ± 0.14 | 0.24 ± 0.03 |
| (mg/cm²) | | |

^{c}The dose was calculated as the difference between unused and used microneedle patches or pilocarpine discs.

The Examples demonstrate that microneedle patches are able to deliver pilocarpine to skin to induce sweating. The amount of sweat produced per dose of pilocarpine delivered, and the chloride concentration of that sweat, were similar for delivery of pilocarpine via microneedle patch and iontophoresis. Thus, microneedle patch delivery is a suitable alternative to iontophoresis delivery of pilocarpine. The pilocarpine dose delivered per unit area doubled with microneedle patch delivery compared to iontophoresis delivery. Therefore, a larger microneedle patch could produce larger amounts of sweat and/or adequate amounts of sweat in less time compared to current iontophoretic methods.

### EXEMPLARY EMBODIMENTS

Embodiment 1. A method for inducing sweat secretion from a patient's skin, comprising: applying a microneedle patch, which comprises microneedles which comprise a sweat-inducing agent, to the skin of the patient effective to cause the microneedles to penetrate across the epidermis and into the dermis; and releasing the sweat-inducing agent into the skin in an amount effective to induce secretion of sweat from the skin.
Embodiment 2. The method of embodiment 1, wherein the sweat-inducing agent is a cholinergic agonist.
Embodiment 3. The method of embodiment 1 or 2, wherein the sweat-inducing agent comprises pilocarpine.
Embodiment 4. The method of any one of embodiments 1 to 3, wherein the applying a microneedle patch comprises manually pressing the microneedle patch against the patient's skin.
Embodiment 5. The method of any one of embodiments 1 to 4, further comprising removing the microneedle patch from the skin after a period of time effective to release the sweat-inducing agent from the microneedle patch into the patient's skin.
Embodiment 6. The method of any one of embodiments 1 to 5, wherein the microneedles are dissolvable microneedles, coated microneedles, or porous/hollow microneedles.
Embodiment 7. The method of embodiment 5 or 6, wherein the period is between 1 second and 15 minutes, e.g., 5 minutes.
Embodiment 8. The method of any one of embodiments 1 to 7, further comprising removing the microneedle patch from the skin in a manner effective to separate the microneedles from a support layer of the microneedle patch, the separated microneedles remaining in the patient's skin and dissolving to release the sweat-inducing agent.
Embodiment 9. The method of any one of embodiments 1 to 8, wherein at least 250 μg of the sweat-inducing agent is delivered per cm² of skin.
Embodiment 10. The method of any one of embodiments 1 to 9, wherein the microneedle patch comprises a support layer from which an array of the microneedles extend.
Embodiment 11. The method of any one of embodiments 1 to 10, wherein the microneedles comprise a water-soluble matrix material in which the sweat-inducing agent is dispersed.
Embodiment 12. The method of embodiment 11, wherein the matrix material comprises a poly(vinyl alcohol) (PVA), a disaccharide, or a combination thereof
Embodiment 13. The method of embodiment 11 or 12, wherein the matrix material comprises PVA and sucrose.
Embodiment 14. The method of any one of embodiments 1 to 13, wherein the microneedles comprise from 30% to 50% by weight of the sweat-inducing agent.
Embodiment 15. The method of any one of embodiments 1 to 14, further comprising collecting and/or analyzing the sweat secreted from the skin.
Embodiment 16. The method of embodiment 15, wherein the collecting of the sweat comprises applying an absorbent material to the skin and/or by positioning a collection tube at the skin surface to permit sweat to be drawn into a bore in the tube by capillary action.
Embodiment 17. The method of embodiment 15 or 16, wherein the volume of sweat collected is at least 15 μl.
Embodiment 18. The method of any one of embodiments 15 to 17, wherein the sweat collected per area of skin into which the sweat-inducing agent is released is at least 2.6 μl per cm².
Embodiment 19. The method of any one of embodiments 15 to 18, wherein the analyzing comprises measuring the sweat for an analyte indicative of cystic fibrosis.
Embodiment 20. The method of any one of embodiments 15 to 19, wherein the analyzing comprises measuring the chloride concentration in the sweat.
Embodiment 21. The method of any one of embodiments 1 to 20, used in the diagnosis of cystic fibrosis.
Embodiment 22. A microneedle patch comprising: a support layer; and an array of microneedles extending from the support layer, wherein the microneedle patch is configured for application to a patient's skin and the microneedles comprise a sweat-inducing agent.
Embodiment 23. The microneedle patch of embodiment 22, wherein the sweat-inducing agent comprises a cholinergic agonist.
Embodiment 24. The microneedle patch of embodiment 22 or 23, wherein the sweat-inducing agent comprises pilocarpine.
Embodiment 25. The microneedle patch of any one of embodiments 22 to 24, wherein the microneedles comprise a water-soluble matrix material in which the sweat-inducing agent is dispersed.
Embodiment 26. The microneedle patch of embodiment 25, wherein the matrix material comprises a poly(vinyl alcohol) (PVA), a disaccharide, or a combination thereof.
Embodiment 27. The microneedle patch of embodiment 25 or 26, wherein the matrix material comprises PVA and sucrose.
Embodiment 28. The microneedle patch of any one of embodiments 22 to 27, wherein the microneedles have a length between 200 μm and 2,000 μm.
Embodiment 29. The microneedle patch of any one of embodiments 22 to 27, wherein the microneedles have a length between 500 μm and 1,000 μm.
Embodiment 30. The microneedle patch of any one of embodiments 22 to 29, wherein each of the microneedles has a base end and an opposing tip end, and wherein the microneedle patch further comprises base pedestals between and connecting the support layer and each of the microneedles.
Embodiment 31. The microneedle patch of embodiment 30, wherein the base pedestals have a height between 200 μm and 800 μm.
Embodiment 32. The microneedle patch of any one of embodiments 22 to 31, wherein the microneedles comprise from 30% to 50% by weight pilocarpine nitrate.
Embodiment 33. The microneedle patch of any one of embodiments 22 to 32, which is configured to deliver at least 250 μg of pilocarpine per cm² of patient's skin.
Embodiment 34. A diagnostic method comprising: inducing secretion of sweat from a patient's skin according to the method of any one of embodiments 1 to 21; and analyzing the sweat for the presence, absence, or concentration of one or more analytes.
Embodiment 35. A medicament comprising pilocarpine for use in the inducement of sweating by administering pilocarpine to the skin of a patient effective to induce secretion of sweat from the skin, wherein the pilocarpine is released into the skin from microneedles applied to the skin of the patient to cause the microneedles to penetrate across the epidermis and into the dermis.
Embodiment 36. The medicament of embodiment 35, wherein the microneedles are dissolvable microneedles, coated microneedles, or porous/hollow microneedles.
Embodiment 37. The medicament of embodiment 35 or 36, wherein at least 250 μg of pilocarpine is delivered per cm² of skin.
Embodiment 38. The medicament of any one of embodiments 35 to 37, wherein the microneedles are in array extending from a support layer of a microneedle patch.
Embodiment 39. The medicament of any one of embodiments 35 to 38, wherein the microneedles comprise a water-soluble matrix material in which the pilocarpine is dispersed.
Embodiment 40. The medicament of embodiment 39, wherein the matrix material comprises a poly(vinyl alcohol) (PVA), a disaccharide, or a combination thereof.
Embodiment 41. The medicament of embodiment 39 or 40, wherein the matrix material comprises PVA and sucrose.
Embodiment 42. The medicament of any one of embodiments 35 to 41, wherein the microneedles have a length between 200 μm and 2,000 μm.
Embodiment 43. The medicament of any one of embodiments 35 to 41, wherein the microneedles have a length between 500 μm and 1,000 μm.
Embodiment 44. The medicament of any one of embodiments 35 to 43, wherein each of the microneedles has a base end and an opposing tip end, and wherein the microneedle patch further comprises base pedestals between and connecting the support layer and each of the microneedles.
Embodiment 45. The medicament of embodiment 44, wherein the base pedestals have a height between 200 μm and 800 μm.
Embodiment 46. The medicament of any one of embodiments 35 to 45, wherein the microneedles comprise from 30% to 50% by weight pilocarpine nitrate.
Embodiment 47. A diagnostic method comprising: inducing secretion of sweat from a patient's skin using the medicament of any one of embodiments 35 to 46; and then analyzing the secreted sweat for the presence, absence, or concentration of one or more analytes.
Embodiment 48. The microneedle patch of any one of embodiments 22 to 33, wherein the microneedles are dissolvable microneedles, coated microneedles, or porous/hollow microneedles.
Embodiment 49. The method of any one of embodiments 1 to 21, wherein 1.5 mg or more of pilocarpine is administered into the skin.
Embodiment 50. The microneedle patch of any one of embodiments 22 to 33 or 48, which is configured to deliver 1.5 mg or more of pilocarpine into the skin.

Modifications and variations of the methods and devices described herein will be obvious to those skilled in the art from the foregoing detailed description. Such modifications and variations are intended to come within the scope of the appended claims.

1. A method of diagnosis of cystic fibrosis in a patient, comprising:
applying a microneedle patch, which comprises dissolvable microneedles which comprise pilocarpine, to the skin of the patient effective to cause the microneedles to penetrate across the epidermis and into the dermis;
releasing the pilocarpine into the skin in an amount effective to induce secretion of sweat from the skin;
collecting a volume of the sweat secreted from the skin; and
analyzing the collected sweat for an analyte indicative of cystic fibrosis.

2. The method of claim 1, wherein the applying a microneedle patch comprises manually pressing the microneedle patch against the patient's skin.

3. The method of claim 1, further comprising removing the microneedle patch from the skin after a period of time effective to release the pilocarpine from the microneedle patch into the patient's skin.

4. (canceled)

5. The method of claim 3, wherein the period is between 1 second and 15 minutes.

6. The method of claim 1, further comprising removing the microneedle patch from the skin in a manner effective to separate the microneedles from a support layer of the microneedle patch, the separated microneedles remaining in the patient's skin and dissolving to release the pilocarpine.

7. The method of claim 1, wherein at least 250 μg of pilocarpine is delivered per cm² of skin.

8. The method of claim 1, wherein the collecting of the sweat comprises applying an absorbent material to the skin or by positioning a collection tube at the skin surface to permit sweat to be drawn into a bore in the tube by capillary action.

9. The method of claim 1, wherein the volume of sweat collected is at least 15 μl.

10. The method of claim 9, wherein the sweat collected per area of skin into which the pilocarpine is released is at least 2.6 μl per cm².

11. The method of claim 1, wherein the microneedle patch comprises a support layer from which an array of the microneedles extend.

12. The method of claim 11, wherein the microneedles comprise a water-soluble matrix material in which the pilocarpine is dispersed.

13. The method of claim 12, wherein the matrix material comprises a poly(vinyl alcohol) (PVA), a disaccharide, or a combination thereof.

14. The method of claim 13, wherein the matrix material comprises PVA and sucrose.

15. The method of claim 12, wherein the microneedles comprise from 30% to 50% by weight pilocarpine nitrate.

16. The method of claim 1, wherein the analyzing comprises measuring the chloride concentration in the collected sweat.

17-19. (canceled)

20. A microneedle patch comprising:
a support layer; and
an array of dissolvable microneedles extending from the support layer,
wherein the microneedle patch is configured for application to a patient's skin and the microneedles comprise a cholinergic agonist.

21. The microneedle patch of claim 20, wherein the cholinergic agonist comprises pilocarpine.

22. The microneedle patch of claim 20, wherein the microneedles comprise a water-soluble matrix material in which the cholinergic agonist is dispersed.

23. The microneedle patch of claim 22, wherein the matrix material comprises a poly(vinyl alcohol) (PVA), a disaccharide, or a combination thereof.

24. The microneedle patch of claim 23, wherein the matrix material comprises PVA and sucrose.

25. (canceled)

26. The microneedle patch of claim 20, wherein the microneedles have a length between 500 μm and 1,000 μm.

27. The microneedle patch of claim 20, wherein each of the microneedles has a base end and an opposing tip end, and wherein the microneedle patch further comprises base pedestals between and connecting the support layer and each of the microneedles.

28. The microneedle patch of claim 27, wherein the base pedestals have a height between 200 μm and 800 μm.

29. The microneedle patch of claim 20, wherein the microneedles comprise from 30% to 50% by weight pilocarpine nitrate.

30. The microneedle patch of claim 29, which is configured to deliver at least 250 μg of pilocarpine per cm² of patient's skin.

31-59. (canceled)
